(19) United States
(12) Patent Application Publication
(10) Pub. No.: US 2022/0168538 A1
(43) Pub. Date: Jun. 2, 2022

(54) Aromatherapy humidifier

(71) Applicant: Dongguan Minghuayuan Craft Products Co., Ltd, Guangdong Province (CN)

(72) Inventor: Shibo Liu, Guang'an City (CN)

(21) Appl. No.: 17/106,212

(22) Filed: Nov. 30, 2020

(51) Int. Cl. A61M 21/00; A61M 11/02; A61L 2/10

(52) U.S. Cl. CPC A61M 21/00; A61M 11/02; A61L 2/10; A61M 2205/362; A61M 2205/75; A61M 2205/3389; A61M 2205/587; A61M 2021/0016

(57) ABSTRACT

The invention discloses an aromatherapy humidifier, which includes a base, a shell and a cover, which are superimposed sequentially. An atomizing container is arranged on the base. A water storage tank is arranged on the atomizing container. The atomizing container and the water storage tank are contained in the shell. The water storage tank supplies water for the atomizing container and is provided with a mist channel communicated with the atomizing container. The atomizing container is provided with an atomizing assembly for atomizing water into mist and an aromatherapy assembly for volatilizing essential oil. The cover is covered on the shell and the water storage tank, the cover is provided with a spray port communicated with the mist channel and an water inlet communicated with the water storage tank, the mist and the volatilizing essential oil float through the mist channel and the spray port to humidify air.

### TECHNICAL FIELD

The present invention relates to humidifier technical field, more particularly, to aromatherapy humidifier.

### BACKGROUND

As the development of science and technology, people's living standards are improving day by day, and people's requirements for living environment are gradually improving. Air humidity is one of the important factors affecting environmental comfort. Especially at the north part of China and the place where air conditioning is used for a long time, the environment is very dry. It is necessary to use the corresponding equipment to increase the humidity of the air and adjust the comfortable living environment. Therefore, aromatherapy humidifier is more and more popular in the public family and become a necessary home appliances.

At present, the general humidifier is provided with an atomizer to atomize water, some essential of is added into the water to atomize with it. However, the way of mixing essential oil and water in a certain proportion has many disadvantages, for example, in order to smell in the mist, the amount of essential oil is higher which increases the cost, and the essential oil is mostly acidic, it is easy to corrode the ordinary plastic container, which makes the life of humidifier shorter.

### SUMMARY

In order to over come the shortcomings of the prior art, the present invention provides an aromatherapy humidifier, which separate the essential oil and the water to be atomized in the atomizing chamber to solve the problem of high consumption of essential oil and corrosion to plastic containers. The present invention also improves the structure of the aromatherapy humidifier, which can automatically control the water storage in the atomizing chamber. The structure of aromatherapy humidifier is more reasonable and simple.

The technical scheme of the present invention is as follows:

An aromatherapy humidifier includes a base, a shell and a cover, which are superimposed sequentially. An atomizing container is arranged on the base. A water storage tank is arranged on the atomizing container. The atomizing container and the water storage tank are contained in the shell. The water storage tank supplies water for the atomizing container and is provided with a mist channel communicated with the atomizing container. The atomizing container is provided with an atomizing assembly for atomizing water into mist and an aromatherapy assembly for volatilizing essential oil. The cover is covered on the shell and the water storage tank, the cover is provided with a spray port communicated with the mist channel and an water inlet communicated with the water storage tank, the mist and the volatilizing essential oil float through the mist channel and the spray port to humidify air.

The atomizing container is a groove formed by a bottom plate and a side plate.

The atomizing assembly is arranged on an atomizing bottom plate, which are sealed at the side surface of the bottom plate facing the base, and the atomizing assembly is passed through the bottom plate into the atomizing container.

A part of the bottom plate further sinks to form the atomizing chamber, which is provided with a first through hole communicating with the base. The atomizing bottom plate is fixed at the side of the atomizing chamber towards the base, the atomizing bottom plate seals the first through hole, and the atomizing assembly extends through the first through hole into the atomizing chamber.

The bottom plate is provided with a sink, an aromatherapy assembly, such as an essential oil bottle, is contained in the sink.

The other part of the bottom plate is rose to form a platform, the sink is arranged at the platform, thereby the aromatherapy assembly is higher than the water after it is contained in the sink.

Preferably, a side of the sink is provided with a wind channel, the side of the bottom plate facing the base is provided with an air-blower, the air-blower blows wind towards the wind channel, the wind channel is provided with wind holes at the side toward the aromatherapy assembly. The wind passes through the wind holes to blow the essential oil to volatilize.

The atomizing container is provided with a control-water assembly for controlling the water in the water storage tank into the atomizing container. The control-water assembly includes a bracket, a floating plate, an ejector rod and a sealing plug. The bracket is fixedly arranged on the bottom plate, the floating plate has a main body and an installing portion arranged on the main body, the installing portion pivots a rotary shaft, the rotary shaft is fixedly arranged on the bracket. The side of the installing portion towards the water storage tank is provided with a pushing column. The top end of the pushing column is provided an inclined surface. The bottom end of the ejector rod is matched with the inclined surface.

Preferably, the bottom end of the ejector rod is a matching plate which is close to the inclined surface. The top end of the ejector rod is provided with the sealing plug. The bottom of the water storage tank is provided with a second through hole. The top end of the ejector rod extends into the second through hole and the sealing plug seals it.

Preferably, the bottom plate of the atomizing container sinks to form a water chamber, and the floating plate and the bracket are housed in the water chamber.

Preferably, the water storage tank is provided with a filter at the second through hole to clean and filter the water entering the atomizing chamber.

Preferably, the water chamber and the atomizing chamber are communicated through a water channel, in which an ultraviolet lamp is arranged, the wall of the water channel is also provided with silica gels around the ultraviolet lamp, and the top of the ultraviolet lamp is provided with a lamp cover.

Preferably, the atomizing chamber is provided with a control-water assembly for controlling the water in the water storage tank into the atomizing chamber. The control-water assembly includes a motor arranged at the bottom plate of the atomizing container, the motor has a shaft which is provided with a horizontal commutator, the horizontal commutator is provide with a vertical pushing column, the vertical pushing column is provided with a seal, the vertical pushing column passes through the bottom plate of the atomizing chamber to support an ejector rod, the seal seals the place where the vertical pushing column passes through the bottom plate. The top of the ejector rod is fixedly provided with a sealing plug , the bottom of the water storage tank is provided with a second through hole. The top end of the ejector rod extends into the second through hole, the sealing plug seals the second through hole.

The control-water assembly also has a water level detector arranged on the bottom plate, the water level detector passes through the bottom plate and extends into the atomizing container, the water level detector is electrically connected and signally connected with a control board.

The end of the horizontal commutator is provided with a spring. The bottom plate is provided with a position column, the spring is arranged on the positioning column.

The water storage tank has a bottom plate above the atomizing chamber, the mist channel is arranged on the bottom plate.

Preferably, the mist channel has an mist inlet at its bottom and an mist outlet at its top, a baffle plate is also arranged at the mist inlet and the mist outlet respectively. The baffle plate is provided with pores for the mist passing through.

The cover is covered on the shell and the water storage tank. The cover has an ring portion and a center portion, The ring part is a downward slope, and the center portion is supported on the ring portion through several supporting rods. The water inlet is formed among the ring portion, the center portion and the supporting rods. The center portion is provided with the spray port toward the mist channel The mist is sprayed out from the spray port.

The base is provided with a button and a power supply socket, and the base is provided with a control board in it. The button is electrically connected with the control board, the control board is electrically connected with the atomizing assembly, the ultraviolet lamp, the air-blower to control them to work or stop working. The power supply socket is connected with a power supply.

The base is also provided with a plurality of LED lights, which are illuminated from the bottom of the aromatherapy humidifier to make the aromatherapy humidifier more beautiful when used. Preferably, the LED is a multi-color lamp, when used, it changes the colors in turns. the LED lamp is electrically connected with the control board.

The aromatherapy humidifier according to the present invention has the advantages: The aromatherapy humidifier is provided with the water chamber and the sink for containing the essential oil in the atomizing chamber. The essential oil is mixed with the mist, and the consumption of the essential oil is less to avoid corrosion to the atomizing chamber made from plastic material, thus prolonging the service life of the aromatherapy humidifier.

The aromatherapy humidifier is also provide with a control-water assembly to automatically control the water amount in the atomizing chamber.

The aromatherapy humidifier is also provide with the ultraviolet lamp to sterilize the water in the atomizing chamber to ensure that the atomized water is clean and hygienic.

The aromatherapy humidifier is also provide with LED lamps at its bottom, which makes the aromatherapy humidifier show dynamic light change effect and beautiful shape.

### BRIEF DESCRIPTION OF THE DRAWINGS

In order to explain more clearly the technical scheme in the embodiment of the present invention, a brief introduction is given below to the attached drawings needed in the embodiment. Obviously, the attached drawings in the following description are only some embodiments of the present invention, for ordinary technicians in this field, other drawings can be obtained according to these drawings without paying creative labor.

FIG. 1 is a perspective view of the aromatherapy humidifier according to the present invention.

FIG. 2 shows the decomposition view of the aromatherapy humidifier shown in FIG. 1.

FIG. 3 is a top view of the aromatherapy humidifier shown in FIG. 1.

FIG. 4 is an section view along A-A line in FIG. 3.

FIG. 5 is a schematic diagram of the base and the atomizing chamber of the aromatherapy humidifier.

FIG. 6 is a decomposition view of FIG. 5.

FIG. 7 is a top view of FIG. 6.

FIG. 8 is an section view along A-A line in FIG. 7.

FIG. 9 is a perspective view of the the atomizing chamber.

FIG. 10 is the other top view of the aromatherapy humidifier shown in FIG. 1.

FIG. 11 is an section view along B-B line in FIG. 10.

FIG. 12 shows the perspective view of the control-water assembly in the other embodiment.

### DETAILED DESCRIPTION OF EMBODIMENTS

The embodiment of the present invention is described in detail below, and an example of the embodiment is shown in the attached Figs., where the same or similar label from beginning to end represents the same or similar element or element with the same or similar function. The following embodiments described by reference to the attached drawings are exemplary and are intended to be used to explain the embodiments of the present invention, and can not be understood as limiting the present invention.

In the description of the embodiment of the present invention, it needs to be understood that the azimuth or position relation indicated in the term “length”, “width”, “upper”, “lower”, “front”, “rear”, “left”, “right”, “vertical”, “horizontal”, “top”, “bottom”, “inside”, “outside” and so on is based on the azimuth or position relation shown in the attached figure, only for the convenience of describing the embodiment and simplifying the description of the present invention, rather than indicating or implying that the device or element must have a specific orientation, construction and operation with a specific orientation, so it can not be understood as a restriction on the present invention.

Furthermore, the terms “first”, “second” are used only for descriptive purposes and can not be understood as indicating or implying relative importance or implicitly indicating the number of technical features indicated. Thus, the qualification of “first” and “second” features may include one or more of them explicitly or implicitly. In the description of the embodiment of the present invention, the meaning of “multiple” is two or more, unless otherwise specified.

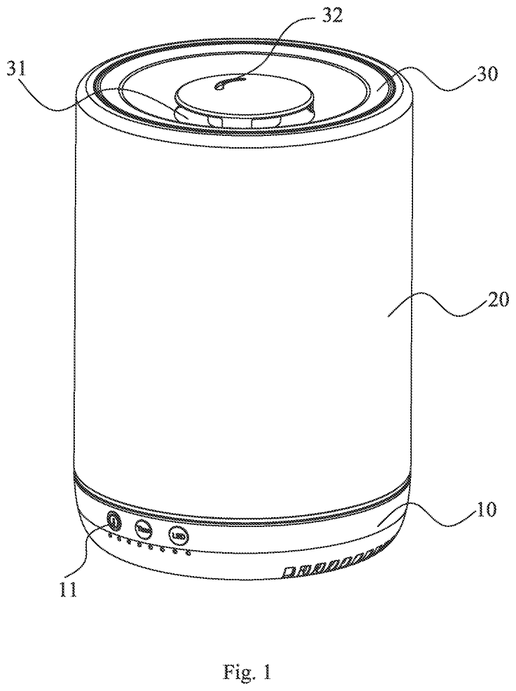

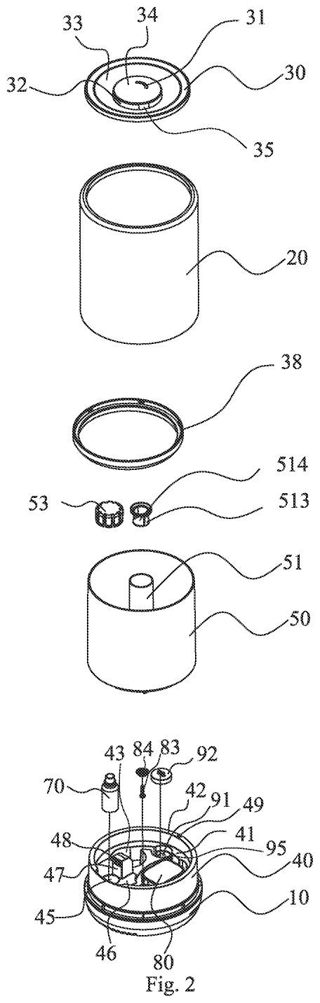

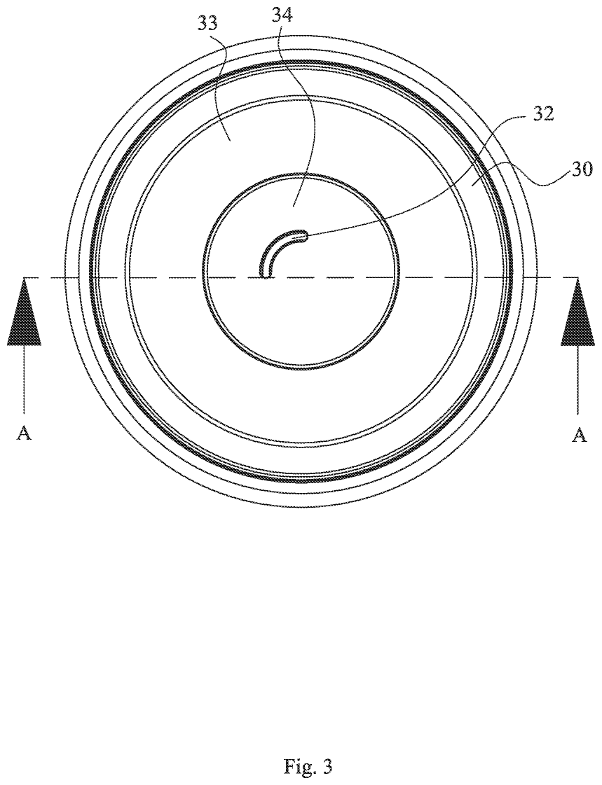

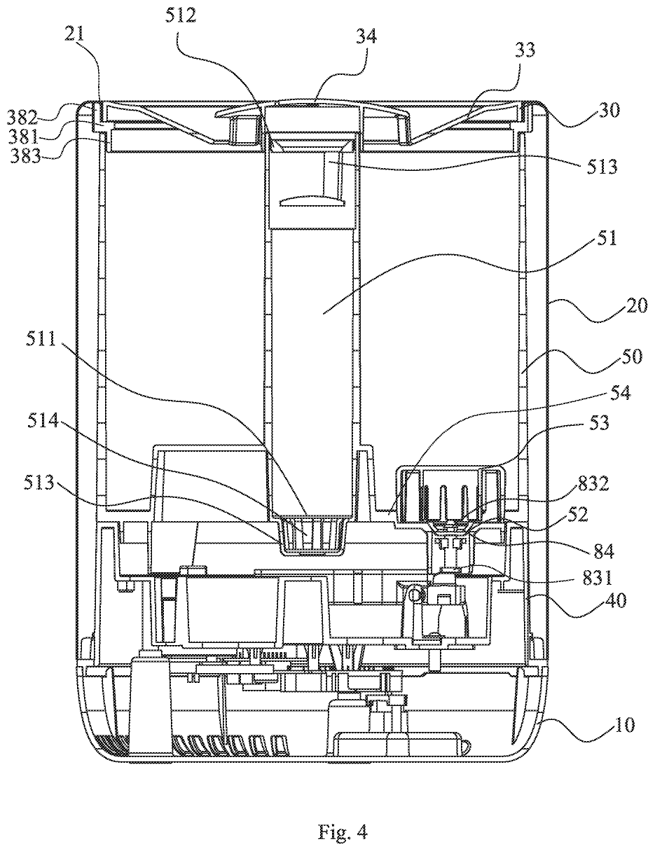

Referring to FIG. 1, FIG. 2, FIG. 3 and FIG. 4, an aromatherapy humidifier includes a base 10, a shell 20 and a cover 30, which are superimposed sequentially. An atomizing container 40 is arranged on the base 10. A water storage tank 50 is arranged on the atomizing container 40. The atomizing container 40 and the water storage tank 50 are contained in the shell 20. The water storage tank 50 supplies water for the atomizing container 40 and is provided with a mist channel 51 communicated with atomizing container 40. The atomizing chamber 40 is provided with an atomizing assembly 60 for atomizing water into mist and an aromatherapy assembly 70 for volatilizing essential oil. The cover 10 is covered on the shell 20 and the water storage tank 50, the cover 30 is provided with a spray port communicated with the mist channel 51 and an water inlet communicated with the water storage tank 50, the mist and the volatilizing essential oil float through the mist channel 51 and the spray port and humidify air.

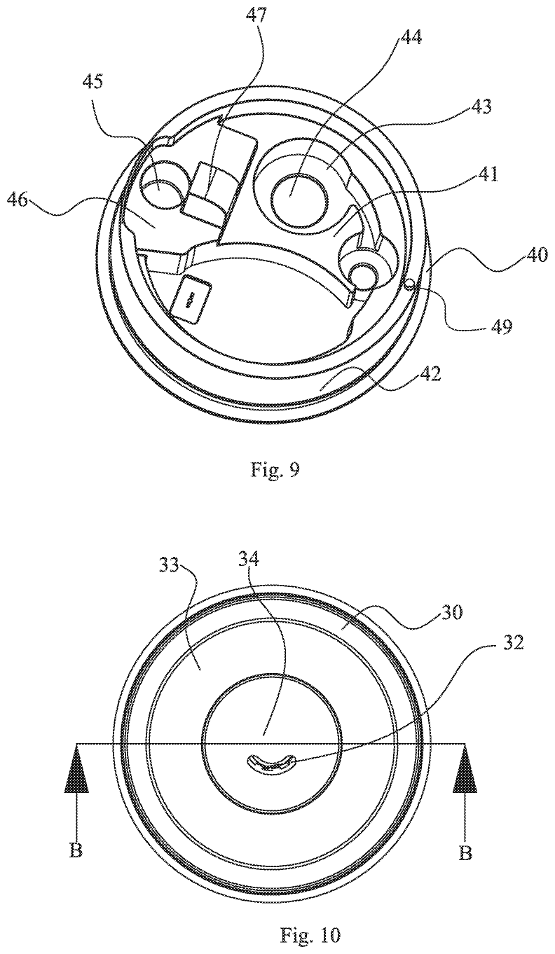

Referring to FIG. 2, FIG. 4 and FIG. 9, the atomizing container 40 is a groove formed by a bottom plate 41 and a side plate 42.

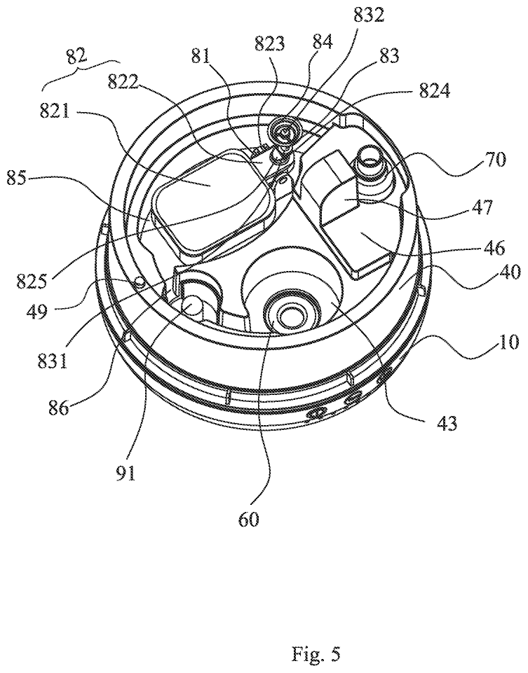

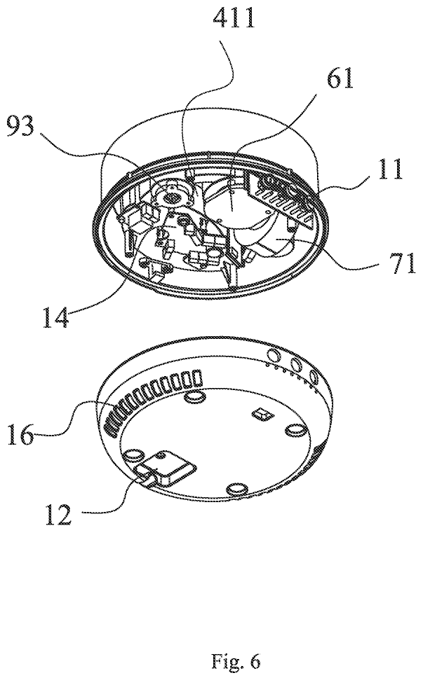

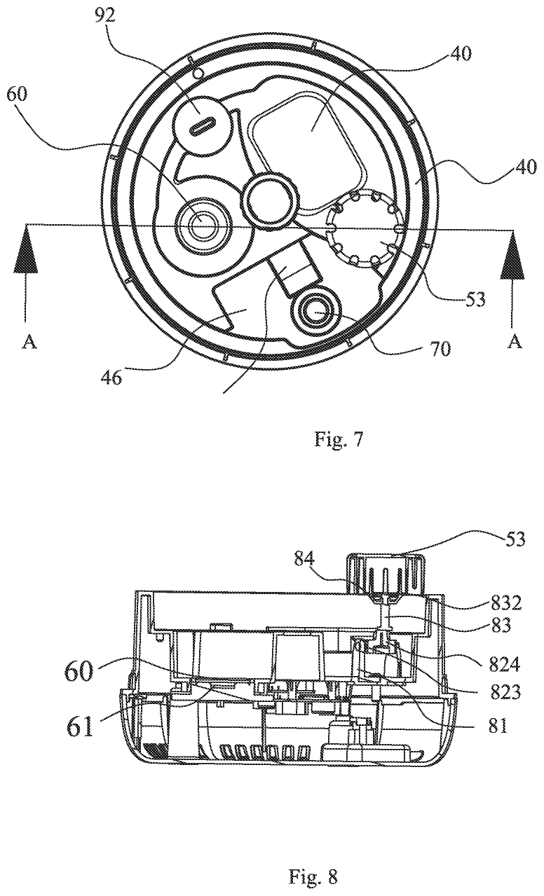

Referring to FIG. 5, FIG. 6, FIG. 8 and FIG. 9, the atomizing assembly 60 is arranged on an atomizing bottom plate 61, which are sealed at the side surface 411 of the bottom plate 41 facing the base 10, and the atomizing assembly 60 is passed through the bottom plate 41 into the atomizing container 40. Concretely, a part of the bottom plate 41 further sinks to form an atomizing chamber 43, which is provided with a first through hole 44 communicating with the base 10. The atomizing bottom plate 61 is fixed at the side of the atomizing chamber 43 towards the base 10 and seals the first through hoe 44, and the atomizing assembly 60 is provided on the atomizing bottom plate 61 and extends through the first through hole 44 into the atomizing chamber 43.

Referring to FIG. 2 and FIG. 5, the bottom plate 41 is provided with a sink 45, an aromatherapy assembly 70, such as an essential oil bottle, is contained in the sink 45. Preferably, in order to prevent the water in the atomizing container 40 from entering the aromatherapy assembly 70, the other part of the bottom plate 41 rises to form a platform 46, and the sink 45 is arranged on the platform 46, thereby the aromatherapy assembly is higher than the water after it is contained in the sink, and the water is prevent from entering the aromatherapy assembly 70.

Referring to FIG. 2, FIG. 5 and FIG. 6, preferably, a side of the sink 45 is provided with a wind channel 47, The side of the bottom plate 41 facing the base 10 is provided with an air-blower 71, the air-blower 71 blows wind towards the wind channel 47, the wind channel 47 is provided with wind holes 48 at the side toward the sink 45. The wind passes through the wind holes 48 to blow the essential oil to volatilize. Concretely, the wind channel 47 extends into the base 10, the air-blower 71 blows towards it.

Referring to FIG. 4, FIG. 5 and FIG. 8, the atomizing container 40 is provided with a control-water assembly 80 for controlling the water in the water storage tank 50 into the atomizing container 40.

In some embodiments of the invention, the control-water assembly 80 includes a bracket 81, a floating plate 82, an ejector rod 83 and a sealing plug 84. The bracket 81 is fixedly arranged on the bottom plate 41, the floating plate 82 has a main body 821 and an installing portion 822 arranged on the main body 821, the installing portion 822 pivots a rotary shaft 823, the rotary shaft 823 is fixedly arranged on the bracket 81. The side of the installing portion 822 towards the water storage tank 50 is provided with a pushing column 824. The top of the pushing column 824 is provided an inclined surface 825. The bottom end of the ejector rod 83 is matched with the inclined surface 825.

Concretely, the bottom end 831 of the ejector rod 83 is a matching plate which is close to the inclined surface 825. The top end 832 of the ejector rod 83 is provided with the sealing plug 84. The bottom 54 of the water storage tank 50 is provided with a second through hole 52. The top end 832 of the ejector rod 83 extends into the second through hole 52 and the sealing plug 84 seals it. When the water in the atomizing container is enough, under the buoyancy of water, the floating plate 82 is raised relative to the installing portion 822, the inclined surface 825 of the pushing column 824 slips relative to the bottom end 831 of the ejector rod 83 from the higher place to the lower place on the inclined surface 825, and the ejector rod 83 drops under the action of gravity. The sealing plug 84 then drops and seals the second hole 52. The water in the water storage cylinder 50 can no longer flow into the atomizing container 40.

When the water in the atomizing container is not enough, the buoyancy suffering by the floating plate 82 is reduced, so the floating plate 82 is reduced relative to the installing portion 822, the inclined surface 825 of the pushing column 824 slips relative to the bottom end 831 of the ejector rod 83 from the lower place to the higher place on the inclined surface 825, and the ejector rod 83. The sealing plug 84 then rises and opens the second hole 52. The water in the water storage cylinder 50 can flow into the atomizing container 40.

Preferably, the bottom plate 41 of the atomizing container 40 sinks to form a water chamber 85, and the floating plate 82 and the bracket 81 are housed in the water chamber 85.

Preferably, the water storage tank 50 is provided with a filter 52 at the second through hole 52 to clean and filter the water entering the atomizing container 40.

It should be understood that the above control-water assembly 80 is not the only structure that can control the water in the water storage tank 50 to enter the atomizing chamber 40. In other embodiments of the invention, another structure of a control-water assembly 80′ is provided.

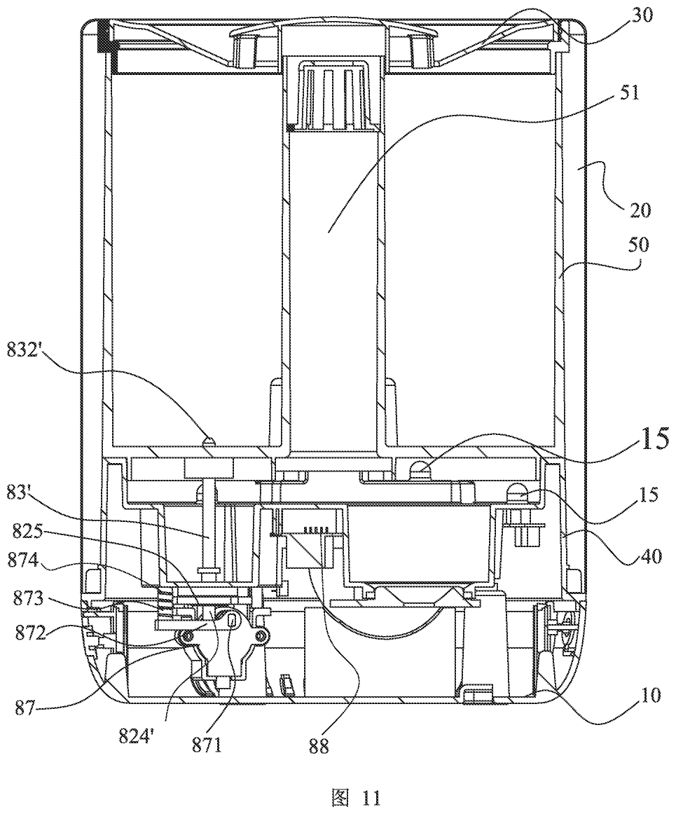

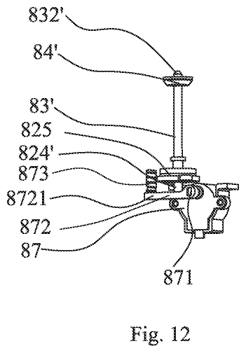

Referring to FIG. 10, FIG. 11 and FIG. 12, the control-water assembly 80′ includes a motor 87 arranged at the bottom plate of the atomizing container 40, the motor 87 has a shaft 871 which is provided with a horizontal commutator 872, the horizontal commutator 872 is provide with a vertical pushing column 824′, the vertical pushing column 824′ is provided with a seal 825, the vertical pushing column 824′ passes through the bottom plate 41 of the atomizing container 40 to support an ejector rod 83′, the seal 825 seals the place where the vertical pushing column 824′ passes through the bottom plate 41. The top end 832′ of the ejector rod 83′ is fixedly provided with a sealing plug 84′, the bottom 54 of the water storage tank 50 is provided with a second through hole 52. The sealing plug 84′ seals the second through hole 52. The motor 87 drives the horizontal commutator 872 to move up and down, thus driving the pushing column 824′ and the ejector rod 83′ to move up and down, thereby open and close the second through hole 52.

The control-water assembly 80′ also has a water level detector 88 arranged on the bottom plate 41, the water level detector 88 passes through the bottom plate 41 and extends into the atomizing container 41, the water level detector 88 is electrically connected and signally connected with a control board 15. When the water level detector 88 detects that the water level in the atomizing container 40 is below a predetermined value, the control board 15 starts the motor 87 to drive the ejector rod 83′ to move up and down, thereby opening the second through hole 52. The water in the water storage cylinder 50 can enter the atomizing container 40. Conversely, When the water level detector 88 detects that the water level in the atomizing container 40 is higher than the predetermined value, The control board 15 closes the motor 87, The sealing plug 84′ seals the second through hole 52, The water storage cylinder 50 stops supplying water to the atomizing container 40. This structure realizes intelligent control of water level in the atomizing container 40 and prevent “dry burning” from happening, improve the safety.

The end 8721 of the horizontal commutator 872 is provided with a spring 873. The bottom plate 41 is provided with a position column 874, the spring 873 is arranged on the positioning column 874. When the motor 87 stops to driving the horizontal commutator 872 to move up and down, the spring 873 adapts to the bottom plate 41 and push the horizontal commutator 872 to reset, thereby the ejector rod 83′ is reset, the sealing plug 84′ seals the second through hole 52. The water in the water storage tank 50 can enter the the atomizing container 40.

Referring to FIG. 5, preferably, the water chamber 85 and the atomizing chamber 43 are communicated through a water channel 86, in which an ultraviolet lamp 91 is arranged, The ultraviolet lamp 91 is used for sterilizing water in the atomizing container. The top of the ultraviolet lamp 91 is provided with a lamp cover, which is used for blocking ultraviolet lights from its top and avoiding injury. A lamp holder 93 is arranged under the ultraviolet lamp 91, and the lamp holder 93 is installed on one side of the bottom plate 41 towards the base 10 and passes through the bottom plate 41. The lamp holder 93 is used to support the ultraviolet lamp 91 and to supply power to it. In order to prevent long time exposure to ultraviolet light and lead to aging of the atomizing container, the water channel 86 is provided with a detachable silica gel member 95 around the ultraviolet lamp 91. The silica gel member 95 is preferably glued to the water channel 86 to facilitate replacement, and to protect the atomizing container and prolong its service life.

Referring to FIG. 2 and FIG. 4, the mist channel 51 is arranged on the bottom 54 of the water storage tank 50, the mist channel 51 communicates through the atomizing container 40, the mist passes through the mist channel 51 and floats out to the environment.

Preferably, the mist channel 51 has an mist inlet 511 at its bottom and an mist outlet 512 at its top, a baffle plate 513 is also arranged at the mist inlet 511 and the mist outlet 512 respectively. The baffle plate 513 is provided with pores 514 for the mist passing through. The baffle is used for blocking the vapour for separate the gathering water in the vapour.

Referring to FIG. 1, FIG. 2 and FIG. 4, the top of the shell 30 and the top of the water storage tank 50 are connected. The cover 30 is covered on the shell 30 and the water storage tank 50. Concretely, a connecting piece 38 is used to connect the top of the shell 20 and the top of the water storage tank 50 and support the shell 30. The connecting piece 38 has has a bearing table 381, an upper connecting portion 382 and a lower connecting portion 383 respectively opposite to be arranged on the upper side and the lower side of the bearing table 381. The top of the shell is provided with a slot 21. The upper connecting portion 382 is inserted into the slot 21. The lower connecting portion 383 is attached to the inner wall of the top of the water storage tank 50. The bearing table 381 lies between the slot 21 and the top of the water storage tank 50. The connecting piece 38 seals the space between the shell 20 and the water storage tank 50 to avoid the mist entering. The covers 30 is placed on the connecting piece 38.

The cover 30 has an ring portion 33 and a center portion 34. The ring part 33 is a downward slope, and the center portion 34 is supported on the ring portion 33 through several supporting rods 35. The water inlet 31 is formed among the ring portion 33, the center portion 34 and the supporting rods 35. When it needs to add water into the water storage tank 50, the water is poured on the ring part 33 and flows through the water inlet 31 into the water storage tank 50. The center portion 34 is provided with the spray port 32 toward the mist channel 51. The mist is sprayed out from the spray port 32.

Referring to FIG. 6, the base 10 is provided with a button 11 and a power supply socket 12, and the base 10 is provided with a control board 14 in it. The button 11 is electrically connected with the control board 14, the control board 14 is electrically connected with the atomizing assembly 60, the ultraviolet lamp 91, the air-blower 71 to control them to work or stop working. The power supply socket 12 is connected with a power supply for atomizing assembly 60, the ultraviolet lamp 91, the air-blower 71.

Referring to FIG. 11, the base 10 is also provided with a plurality of LED lights 15, which are illuminated from the bottom of the aromatherapy humidifier , the shell 20 is made of transparent material capable of transmitting light from the side of the shell 20, which makes the aromatherapy humidifier more beautiful when used. Preferably, the LED 15 is a multi-color lamp, when used, it changes the colors in turns. the LED lamp 15 is electrically connected with the control board 14.

The base 10 is also provided with a wind bore 16 for the blower 71 to blow and radiate heat.

The shell 20 is made of metal material, glass material, ceramic material, plastic material, etc. The atomizing container 40 is made of metal material, glass material, ceramic material, plastic material, etc.

The top of the side plate 42 is provided with a lift switch 49 and a holding slot for the lift switch 49, which controls the control plate 14. When the water storage tank 50 is placed on the atomizing container 40, the lift switch 49 is pressed, thus opening the control board 14 to supply power for the atomizing assembly 60, the ultraviolet lamp 91, and the blower 71. When the water storage tank 50 is removed from the atomizing container 40, the pressing switch 49 rises and disconnects the control board 14 so that it can not supply power for the atomizing container 60, the ultraviolet lamp 91, and the blower 71.

The above contents are further detailed instructions to the utility model in combination with the specific optimal selection implementation mode, and it can not be concluded that the specific implementation of the utility model is limited to these instructions. For the ordinary technical personnel in the technical field of the utility model, the structure form of the utility model can be flexible and changeable, and the series of products can be derived without breaking away from the conception of the utility model. Only a few simple deductions or replacements should be regarded as falling within the scope of patent protection of the utility model determined by the submitted claim.

1. An aromatherapy humidifier includes a base, a shell and a cover, which are superimposed sequentially; an atomizing container is arranged on said base; a water storage tank is arranged on said atomizing container; said atomizing container and said water storage tank are contained in said shell; wherein said water storage tank supplies water for said atomizing container and is provided with a mist channel communicated with said atomizing container; said atomizing container is provided with an atomizing assembly for atomizing water into mist and an aromatherapy assembly for volatilizing essential oil; said cover is covered on said shell and said water storage tank, said cover is provided with a spray port communicated with said mist channel and an water inlet communicated with said water storage tank, said mist and said volatilizing essential oil float through said mist channel and said spray port to humidify air.

2. The aromatherapy humidifier according to claim 1, wherein said atomizing container is a groove formed by a bottom plate and a side plate;
said atomizing assembly is arranged on an atomizing bottom plate, which are fixed at a side surface of said bottom plate towards said base, and said atomizing assembly is passed through said bottom plate into said atomizing container.

3. The aromatherapy humidifier according to claim 2, wherein a part of the bottom plate sinks to form a atomizing chamber, which is provided with a first through hole communicating with said base; said atomizing bottom plate is fixed at a side of said atomizing chamber towards said base, said atomizing bottom plate seals said first through hole, and said atomizing assembly extends through said first through hole into said atomizing chamber.

4. The aromatherapy humidifier according to claim 2, wherein the other part of the bottom plate is rose to form a platform, a sink is arranged at said platform, said aromatherapy assembly is higher than said water after it is contained in said sink.

5. The aromatherapy humidifier according to claim 4, wherein a side of said sink is provided with a wind channel, a side of said bottom plate facing said base is provided with an air-blower, said air-blower blows wind towards said wind channel, said wind channel is provided with wind holes at a side toward said sink, said wind passes through said wind holes to blow said essential oil to volatilize.

6. The aromatherapy humidifier according to claim 1, wherein said atomizing container is provided with a control-water assembly for controlling water in said water storage tank into said atomizing container, said control-water assembly includes a bracket, a floating plate, an ejector rod and a sealing plug, said bracket is fixedly arranged on said bottom plate, said floating plate has a main body and an installing portion arranged on said main body, said installing portion pivots a rotary shaft, said rotary shaft is fixedly arranged on said bracket; a side of said installing portion towards said water storage tank is provided with a pushing column, a top end of said pushing column is provided an inclined surface, a bottom end of said ejector rod is matched with said inclined surface; said bottom end of said ejector rod is a matching plate which is close to said inclined surface, said top end of said ejector rod is provided with said sealing plug; a bottom of said water storage tank is provided with a second through hole, a top end of said ejector rod extends into said second through hole and said sealing plug seals it.

7. The aromatherapy humidifier according to claim 6, wherein said bottom plate of said atomizing container sinks to form a water chamber, and said floating plate and said bracket are housed in said water chamber.

8. The aromatherapy humidifier according to claim 6, wherein said water storage tank is provided with a filter at said second through hole to clean and filter water entering said atomizing chamber.

9. The aromatherapy humidifier according to claim 6, wherein said water chamber and said atomizing chamber are communicated through a water channel, in which an ultraviolet lamp is arranged, said wall of said water channel is also provided with silica gels around said ultraviolet lamp, and a top of the ultraviolet lamp is provided with a lamp cover.

10. The aromatherapy humidifier according to claim 1, wherein said atomizing chamber is provided with a control-water assembly for controlling water in said water storage tank into said atomizing chamber, said control-water assembly includes a motor arranged at a bottom plate of said atomizing container, said motor has a shaft which is provided with a horizontal commutator, said horizontal commutator is provide with a vertical pushing column, said vertical pushing column is provided with a seal, said vertical pushing column passes through a bottom plate of said atomizing chamber to support an ejector rod, said seal seals a place where said vertical pushing column passes through said bottom plate; a top end of the ejector rod is fixedly provided with a sealing plug, a bottom of said water storage tank is provided with a second through hole; said top end of said ejector rod extends into said second through hole , said sealing plug seals said second through hole.

11. The aromatherapy humidifier according to claim 10, wherein said control-water assembly also has a water level detector arranged on said bottom plate, said water level detector passes through said bottom plate and extends into the atomizing container, said water level detector is electrically connected and signally connected with a control board.

12. The aromatherapy humidifier according to claim 10, wherein an end of said horizontal commutator is provided with a spring, said bottom plate is provided with a position column, said spring is arranged on said positioning column.

13. The aromatherapy humidifier according to claim 1, wherein said water storage tank has a bottom plate above said atomizing chamber, said mist channel is arranged on said bottom plate;
said mist channel has an mist inlet at its bottom and an mist outlet at its top, a baffle plate is also arranged at said mist inlet and said mist outlet respectively, said baffle plate is provided with pores for said mist passing through.

14. The aromatherapy humidifier according to claim 1, wherein said cover is covered on said shell and said water storage tank, said cover has an ring portion and a center portion, said ring part is a downward slope, and said center portion is supported on said ring portion through several supporting rods, said water inlet is formed among said ring portion, said center portion and said supporting rods, said center portion is provided with said spray port toward said mist channel, said mist is sprayed out from said spray port.

15. The aromatherapy humidifier according to claim 1, wherein the base is also provided with a plurality of LED lights, the shell is made of transparent material capable of transmitting light from the side of the shell.
